(19) United States
(12) Patent Application Publication
(10) Pub. No.: US 2002/0013566 A1
(43) Pub. Date: Jan. 31, 2002

(54) Absorbent article having a removal aid and a method for removing the absorbent article

(75) Inventors: Charles John Chappell, Appleton, WI (US); Wayne Clement Sigl, Neenah, WI (US)

Correspondence Address: WILHELM LAW SERVICE, S.C., 100 W LAWRENCE ST, THIRD FLOOR, APPLETON, WI, 54911

(73) Assignee: Kimberly-Clark Worldwide, Inc., Neenah, WI

(21) Appl. No.: 09/975,108

(22) Filed: Oct. 10, 2001

Related U.S. Application Data
(63) Continuation of application No. 08/768,162, filed on Dec. 13, 1996.

(51) Int. Cl. A61F 13/15

(52) U.S. Cl. 604/385.13

(57) ABSTRACT

The present invention relates to an absorbent article adapted to be secured to a wearer's body. The absorbent article has a cover disposed toward the body facing surface and a baffle disposed toward the undergarment-facing surface. Positioned between the cover and the baffle is an absorbent material. An adhesive is secured to the body facing surface to hold the absorbent article in intimate contact with the wearer during use. The absorbent article further includes a pocket secured to the baffle for assisting removal of the absorbent article. The pocket has a fixed portion and a medial open portion which may be grasped or a portion of the wearer hand may be inserted to facilitate removal. Advantageously, the open portion of the pocket provides a means for hygienically disposing of the sanitary napkin. The method includes folding the sanitary napkin in half (across the transverse axis) so that the cover is in contact with itself and the transverse ends are substantially aligned. The open portion of the pocket is then rolled over the transverse ends to retain the sanitary napkin in a folded condition.

### FIELD OF THE INVENTION

The present invention relates to disposable absorbent articles adapted to be secured to a wearer's body and utilized for the absorption and containment of body fluids such as menstrual fluid, urine and other body exudates. More particularly, the present invention relates to a removal aid for comfortably disengaging the disposable absorbent article from the wearers body and having an improved disposal means that provides convenient disposal of the used product.

### BACKGROUND OF THE INVENTION

Disposable absorbent articles for the absorption and containment of urine, menses and other body exudates are generally known in the art. Such disposable articles have found particular utility in the fields of infant, child, feminine and adult incontinence hygiene. In the area of feminine care, such disposable absorbent articles have been commercialized in two basic types, the tampon and the sanitary napkin. For the purposes described herein, sanitary napkins include thin absorbent devices such as panty liners and interlabial devices. which may have an absorbency of just a few grams, to the larger maxi and overnight pads designed for absorbing a much greater quantity of body fluids. Present sanitary napkins are unitary, preshaped and prefolded and may be individually packaged for discreteness and hygienic purposes. Their construction is generally comprised of a fluid permeable body side cover; a liquid-impermeable, garment-facing baffle and an absorbent positioned between the cover and the baffle. The sanitary napkin also may include an adhesive disposed on the garment-facing surface of the baffle to secure the sanitary napkin in the crotch region of the wearer's undergarment.

After the sanitary napkin is soiled, it is removed and discarded. Removing a sanitary napkin secured to the undergarment usually involves stripping the sanitary napkin from the crotch area of the undergarment. However, if the sanitary napkin has become adhered to the wearer the sanitary napkin may pull pubic hair and tissue from the woman as it is being removed, causing, in most cases, an extreme amount of discomfort.

A sanitary napkin is disclosed in the patent application having U.S. Ser. No. 08/167,597 filed on Dec. 14, 1993, the entire disclosure of which is incorporated herein by reference and made a part hereof, utilizes a body adhesive to secure the sanitary napkin to the wearer's body. To remove this sanitary napkin, the wearer usually grasps an exposed edge of the sanitary napkin and pulls downward. Depending upon the location of the adhesive, grasping an edge may be difficult. The adhesive may also have body fluids, such as menses or other fluids coating its surface. Desirably, the wearer wants to avoid contacting the body excretions to prevent soiling her hands and possibly her clothing. Touching the adhesive may also contaminate that portion of the adhesive, lessening its adhesive strength for retaining the sanitary napkin against the body if reapplied.

Disposal of a soiled product also presents its own unique problems. Sanitary napkins generally have a fluid-impervious backsheet or baffle that prevents disposal by merely flushing the soiled product down a toilet. Generally, napkin users are faced with the task of disposing of a used napkin in open waste paper baskets, often resorting to wrapping the soiled napkin in tissue before discarding.

Therefore, there is a need for a way to comfortably and hygienically remove such an absorbent article from a wearer's body without soiling the person or her clothing. There is also a need for a sanitary napkin that can be sealed into a compact size for disposal.

### SUMMARY OF THE INVENTION

Briefly, the present invention relates to an absorbent article adapted to be secured to a wearer's body. The absorbent article is also capable of being folded and self-sealed for disposal. The absorbent article has a fluid-permeable cover with a body facing surface and a liquid-impermeable baffle with a garment-facing surface. Positioned between the body facing surface and the garment-facing surface is an absorbent material. An adhesive is secured to the body facing surface to hold the absorbent article in intimate contact with the wearer during use. The absorbent article also includes a pocket device having a fixed portion secured to the baffle and an open portion adapted to assist in disengaging or removing the absorbent article from the wearer's body when removal is desired. The pocket device is utilized by the wearer by grasping and/or inserting at least a portion of one or more fingers into the open portion and pulling the absorbent article away from the wearer's body.

The present invention also provides a method of disposing of the sanitary napkin comprising the steps of folding the sanitary napkin along its transverse centerline or axis so that the body facing side of the napkin is folded upon itself and rolling or folding the open portion of the pocket device over the transverse ends of the sanitary napkin to retain-the sanitary napkin in a folded condition. This effectively encloses the soiled portion of the napkin and keeps the absorbed body fluid from leaking. Moreover, the soiled sanitary napkin can now be easily handled and hygienically discarded without needing to wrap the soiled napkin in tissue paper.

It is a general object of the invention to provide a removal aid for comfortably detaching an adhesively secured absorbent article from a wearer's body. More specifically, it is an object of the invention to provide a removal aid on the absorbent article that permits the wearer to remove it with one hand.

It is another object of the invention to provide a sanitary napkin which is adhesively secured to the body and which incorporates a detaching device positioned on the baffle for protecting the user from contacting body fluids when removing the sanitary napkin.

It is another object of the invention to provide a sanitary napkin having its own disposal means.

These and other objects of the invention will be more readily apparent when considered in reference to the following description when taken in conjunction with the accompanying drawings. The invention resides in the novel combination, arrangement of the parts and methods as substantially described herein and more particularly defined by the claims.

### BRIEF DESCRIPTION OF THE DRAWINGS

FIG. 1 is a top view of an absorbent article which may be secured to the wearer's body during use.

FIG. 2 is a bottom view of the absorbent article.

FIG. 3 is a cross-sectional view of FIG. 1 taken along line 3-3.

FIG. 4 is a cross-sectional view of another embodiment.

FIG. 5 is a cross-sectional view illustrating how the pocket means can be utilized to hygienically dispose of a soiled product.

### DETAILED DESCRIPTION OF THE PREFERRED EMBODIMENTS

The present invention relates generally to a disposable absorbent article which is secured to a wearer's body by a pressure sensitive body adhesive. For convenience of description only, the invention will be described as a catamenial device, i.e. a sanitary napkin, but is not limited thereto. It is to be understood that the invention may be adapted for use in other absorbent articles such as diapers, incontinent devices, adhesive bandages and the like which incorporate some means for adhesively adhering the absorbent article to at least a portion of a wearer's body.

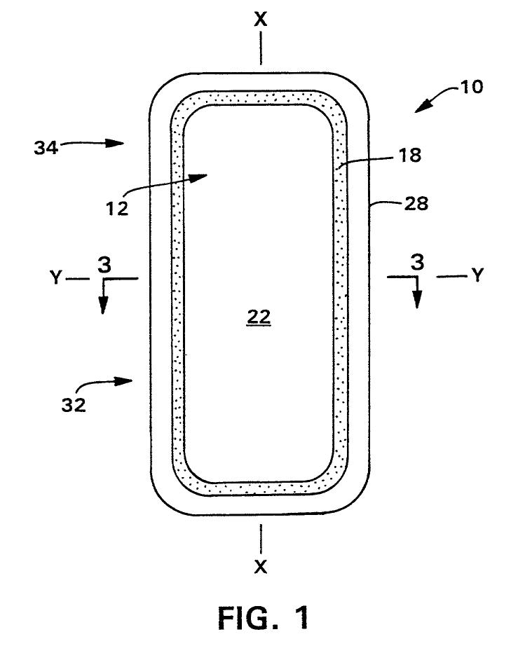

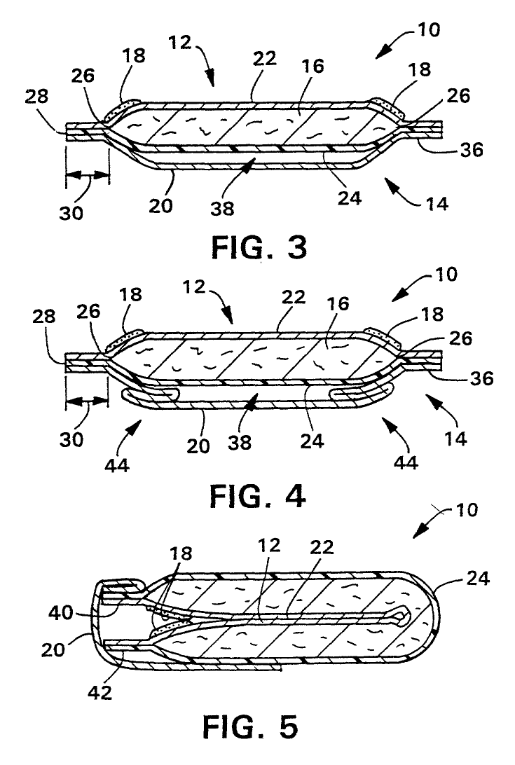

For ease of understanding, when referring to the Figures, the same numeral designates the same or a substantially identical part in the different views and embodiments. Referring to FIGS. 1-5, an embodiment of a sanitary napkin 10 is shown. Broadly, the sanitary napkin 10 includes a fluid-permeable, body facing surface 12; a liquid-impermeable, garment-facing surface 14; an absorbent 16 disposed between the body facing surface 12 and the garment-facing surface 14; and an adhesive 18 secured to the body facing surface 12 for adhering the sanitary napkin 10 to a wearer's body during use. Examples of basic constructed sanitary napkins are disclosed in U.S. patent application Ser. No. 08/475,324 filed Jun. 30, 1995 entitled “ABSORBENT ARTICLE HAVING A THIN, EFFICIENT ABSORBENT CORE” and U.S. patent application Ser. No. 08/716,875 filed Oct. 1,1996 entitled “ABSORBENT ARTICLE HAVING A CELLULOSIC-TRANSFER LAYER” the entire disclosure of each being incorporated herein by reference and made a part hereof.

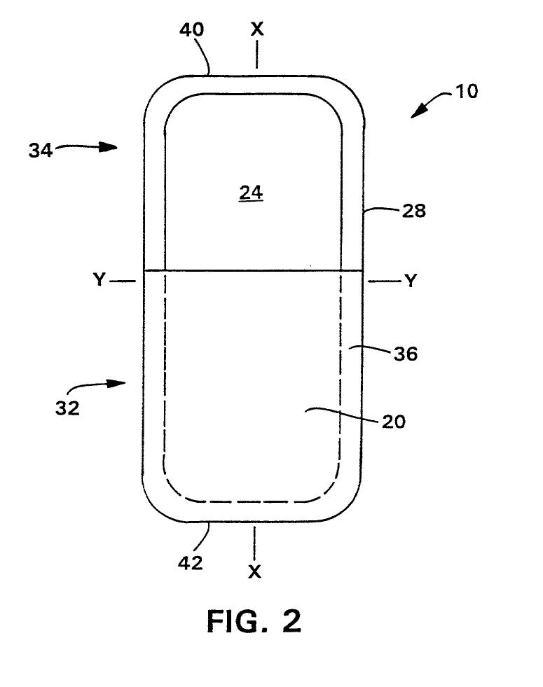

Erring to FIG. 2, a pocket device 20 is disposed toward the garment facing surface 14 of the sanitary napkin 10 to provide an easy way for comfortably disengaging the sanitary napkin 10 from the wearer's body and advantageously provides a way to hygienically dispose of the used product. The sanitary napkin 10 can also include a peel strip, not shown, overlying the adhesive 18 to prevent the adhesive from becoming contaminated and/or prematurely adhering to an unintended surface. Suitable materials for use as a peel strip are well known and commercially available from a number of sources.

As illustrated, the sanitary napkin 10 has a racetrack shape, but is not limited thereto. It can also be hourglass, oval or any other configuration that will allow the sanitary napkin 10 to come into intimate contact with the wearer. As used herein, the term “sanitary napkin” refers to an article which is worn by females adjacent to the pudendal region and which is intended to absorb and contain various exudates which are discharged from the body such as blood, menses, and urine, and which is intended to be discarded when soiled, not laundered and reused. Interlabial devices which reside partially within and partially external of the female wearer's vestibule are also within the scope of this invention.

Referring to FIGS. 1-3, the body facing surface 12 is generally integral to and associated with one surface of a cover 22 and the undergarment-facing surface 14 is generally integral to and associated with one surface of a baffle 24. The cover 22 and baffle 24 can have a length and a width dimension extending in a contiguous relationship beyond an edge 26 of the absorbent 16. The cover 22 and the baffle 24 can be sealed together to define a perimeter 28 of the sanitary napkin 10. The portion from the absorbent edge 26 to the perimeter 28 is defined herein as the peripheral seal 30. The absorbent 16 is thereby enclosed between the cover 22 and the baffle 24. The cover 22 and the baffle 24 may be sealed together using any suitable means that will not leave a hard, uncomfortable residue that may be annoying to the wearer. As used herein, the term “sealed” encompasses configurations whereby the cover 22 is directly joined to baffle 24 and configurations whereby the cover 22 is indirectly joined to the baffle 24 by affixing the cover 22 to an intermediate member, which are in turn affixed to the baffle 24. Methods for attaching the cover 22 and baffle 24 are well known to those skilled in the art and include the use of hot melt adhesives, pressure-sensitive adhesives, double-sided tape, sonic bonding and heat sealing.

The cover 22 is designed to contact the body of the wearer and therefore should be easily penetrated by body fluids. The cover 22 should also be non-irritating to the wearer's skin and preferably will not absorb an appreciable amount of fluid insulting its surface. The cover 22 can be constructed of a woven or nonwoven, natural or synthetic material. Suitable materials include bonded carded webs of polyester, polypropylene, polyethylene, nylon, or other heat-bondable fibers. Other polyolefins, such as copolymers of polypropylene and polyethylene, linear low-density polyethylene, finely-perforated film webs and net material, also work well. Particularly preferred are composite materials of a polymer and a nonwoven fabric material. Still another cover material is a spunbond web of polypropylene. The web can contain about 1% to about 6% titanium dioxide pigment to give it a dean, white appearance. A uniform spunbond material is desirable because it has sufficient strength in the longitudinal direction, even after being perforated, to resist being torn or pulled apart during use. The most preferred polypropylene webs have a weight of between about 18 and 40 grams per square meter. An optimum weight is between about 30 and about 40 grams per square meter.

To aid in the penetration of the liquid through the web, the cover 22 can also be treated with a surfactant to improve its hydrophilic characteristics. The surfactant can include topical additions or internally applied materials like polysiloxanes.

The baffle 24 acts as a barrier between the absorbed body fluids contained in the absorbent 16 and the person wearing the sanitary napkin 10. Accordingly, the baffle 24 is nonabsorbent and is impervious to liquids. The baffle 24 should be soft and compliant since a portion of the baffle 24 may reside adjacent the thigh region of the wearer. As used herein, the term “compliant” refers to materials which will readily conform to the general external shape and contours of the human anatomy. In a preferred embodiment, the baffle 24 may permit the passage of air or vapor out of the sanitary napkin 10 while blocking the passage of liquids from the absorbent 16. A good baffle material is a micro-embossed, polymeric film, such as polyethylene or polypropylene having a thickness in the range of from about 0.012 mm to about 1.0 mm. Bicomponent films can also be used as well as woven and nonwoven fabrics which have been treated to render them liquid-impermeable.

The absorbent 16 is in liquid communication with the body facing surface 12 and is positioned between the body facing surface 12 and the garment-facing surface 14. The term “liquid communication” means that body fluid insulting the body facing surface 12 will, substantially, be absorbed by the absorbent 16. Referring again to FIG. 2, the cover 22 and the baffle 24 in combination enclose the absorbent 16, substantially defining the absorbent's perimeter or edge 26. The materials used in the absorbent 16 are designed to absorb body exudates, including menstrual fluids, blood and urine. Suitable materials include wood pulp fluff, rayon, cotton and meltblown polymer, such as polyester, polypropylene or coform. Coform is an air-formed combination of meltblown polymers, such as polypropylene, and absorbent staple fibers, such as cellulose. A preferred material is wood fluff, for it is low in cost, relatively easy to form and has good absorbency. The absorbent 16 may be a composite comprised of a hydrophilic material that can be formed from various natural or synthetic fibers, wood pulp fibers, regenerated cellulose or cotton fibers, an airlaid tissue or a blend of pulp and other fibers. The absorbent 16 can be made from other well known materials used in absorbent articles, including multiple layers of cellulose wadding, rayon fibers, cellulose sponge, hydrophilic synthetic sponge, such as polyurethane, and the like. The capacity of the absorbent 16 may be varied depending upon the intended usage of the final product.

The sanitary napkin 10 may also include other layers, such as a wicking layer, one or more tissue layers and may contain superabsorbent in combination with one or more other absorbent materials described above, none of which are shown in the Figures. Such layers and materials are commercially available from several sources and are well known to those skill in construction of disposable absorbent articles, such as sanitary napkins, diapers and incontinent devices.

The adhesive 18 is positioned on the body facing surface 12 to contact the wearer and support the sanitary napkin 10 during use. As shown in FIG. 1, the adhesive 18 is adhered to the cover 22. The adhesive 18 can overlie from about 5 percent to about 95 percent of the body facing surface 12. Preferably, the adhesive 18 overlies from about 5 percent to about 75 percent of the body facing surface 12, more preferably, from about 5 percent to about 35 percent and most preferred, from about 5 percent to about 20 percent of the body facing surface 12. Suitable adhesive materials are disclosed in the commonly assigned patent application having U.S. Ser. No. 08/331,072 flied on Oct. 28, 1994, the entire disclosure of which is incorporated herein by reference and made a part hereof.

In an alternative configuration, not shown, the baffle 24 may overwrap a portion of the cover 22 along the absorbent edge 26 so that a portion of the body facing surface 12 would be comprised of the fluid-permeable cover 22 and a portion would be comprised of the liquid-impermeable baffle 24. In this configuration, the adhesive 18 may reside on the cover 22, the baffle 24 or both.

Referring to FIGS. 2-5, the pocket device 20 is positioned adjacent to the garment facing surface 14 of the baffle 24. The pocket device 20 may be formed by securing an appropriately sized separate piece of material to a portion of the baffle 24. Alternatively, the pocket device 20 may be formed by folding back onto the baffle 24 and sealing thereto a portion of excess material used to form the baffle 24. Desirably, the material extends from a transverse end 40 or 42 of the sanitary napkin 10 and is folded back onto the baffle. A portion of the material can then be affixed to the baffle 24 to form the pocket device 20. Any material not used to form the pocket device 20 and which may extend beyond the perimeter 28 could then be removed when the sanitary napkin 10 is shaped via die cutting during its production.

Referring to FIGS. 1 and 2, the sanitary napkin 10 can be divided into substantially equal portions along transverse line Y-Y into a first section 32 and a second section 34. Each section 32 and 34 are of substantially size and have substantially equal surface areas disposed toward the garment facing surface 14. The pocket device 20 has a fixed portion 36 secured to the baffle 24 at or inward of the perimeter 28 of the sanitary napkin 10. As seen in FIG. 2, the fixed portion 36 generally follows the perimeter 28 of the sanitary napkin 10 in that section 32 or 34 in which it is affixed. The pocket device 20 is sized and configured so that it covers greater than about 25 percent of the surface area, preferably, the pocket device 20 covers greater than about 50 percent of the surface area, and more preferably, the pocket device 20 covers greater than about 80 percent of the surface area.

The pocket device 20 also has an open portion 38 that is adapted for assisting the wearer in removing the sanitary napkin 10 from her body. The open portion 38 of the pocket device 20 is sufficient for the wearer to at least partially insert into the open portion 38 one or more of her fingers. Desirably, the open portion 38 extends from the edge 26 of the absorbent 16 to the opposite edge 26 of the absorbent 16, i.e. extending the width of the absorbent 16. The open portion 38 of the pocket device 20 provides a means by which the sanitary napkin 10 can be comfortably and hygienically removed. The wearer can remove or disengage the sanitary napkin 10 from her body by grasping the open portion 38 and/or partially inserting into the open portion 38 a portion of her hand and pulling it directionally away from her body. The pocket device 20 also permits the sanitary napkin 10 to be hygienically handled while toileting or during disposal.

The pocket device 20 may be made from any material that can be affixed to the baffle 24 using any suitable means that will not leave a hard, uncomfortable residue that may be annoying to the wearer. The material should have a modulus greater than the peel strength of the adhesive 18 to prevent the material from tearing during removal. Suitable materials include bonded carded webs of polyester, polypropylene, polyethylene, nylon, or other heat-bondable fibers, polymeric film, such as polyethylene or polypropylene. Desirably, the material used is the same as or similar to the material used in forming the baffle 24. If the baffle 24 is constructed from a breathable film material it is also desirable to construct the pocket device 20 from a compatible breathable material to prevent partially negating the advantage of using a breathable film.

Referring to FIG. 4, the pocket device 20 may further incorporate one or more expanding means 44 for permitting the open portion 38 to expand and/or stretch in at least one direction to accommodate the insertion of several fingers into the pocket. Examples of expanding means includes folds, pleats and using an elastomeric material to form at least a portion of the pocket device 20. The expanding means 44 increases the volume of the open portion 38 by greater than about 5 percent, preferably greater than about 50 percent and more preferably greater than about 80 percent. The elastomeric material should be elastomeric in at least one direction and may have elasticity in both the transverse and longitudinal directions, relative to the sanitary napkin 10. An elastomeric material may be constructed from a polyethylene film laminate having elastic threads incorporated between the laminate material. Material expandability is of further utility in the method of disposal described herein by allowing the open portion 38 of the pocket device 20 to be easily folded over the transverse ends 40 and 42.

Referring to FIGS. 2-5, in the method of the invention, the sanitary napkin 10 is folded about the transverse axis Y-Y so the body facing surface 12 of the cover 22 is folded upon itself, allowing the transverse ends 40 and 42 to be substantially aligned. The open portion 38 of the pocket device 20 is then rolled, folded or pulled over the ends 40 and 42 to hold the ends 40 and 42 together. FIG. 5 shows this enveloping. Thus, the soiled side of the napkin will be enclosed in the folded pad and retained as shown. This will eliminate the need for wrapping the used napkin 10.

The foregoing detailed description has been for the purpose of illustration setting forth the preferred embodiment of the invention. Accordingly, a number of modifications and changes may be made without departing from the spirit and scope of the present invention. Therefore, the invention should not be limited by the specific terms employed, but only by the claims.

We claim:

1. An absorbent article adapted to be adhesively secured to a wearer's body, said absorbent article comprising:
a. a fluid-permeable cover having a body facing surface;
b. a liquid-impermeable baffle having a garment-facing surface;
c. an absorbent positioned between said cover and said baffle;
d. adhesive means for securing said absorbent article to the wearer's body, said adhesive means being affixed to said body facing. surface; and
e. a pocket, device having a fixed portion secured to said baffle and an open portion adapted for assisting removal of said absorbent article.

2. The absorbent article of claim 1 wherein said cover and said baffle extend beyond an edge of said absorbent to form a perimeter of the absorbent article and said fixed portion is secured to said baffle at said perimeter.

3. The absorbent article of claim 1 wherein said cover and said baffle extend beyond an edge of said absorbent to form a perimeter of the absorbent article and said fixed portion is secured to said baffle inward from said perimeter

4. The absorbent article of claim 1 further comprising a transverse axis dividing said absorbent article into a first section and a second section of substantially equal surface area, said pocket device being secured to one of said sections and wherein said pocket device covers greater than about 20 percent of said one section surface area.

5. The absorbent article of claim 4 wherein said pocket device covers greater than about 50 percent of said one section surface area.

6. The absorbent article of claim 4 wherein said pocket device covers greater than about 80 percent of said one section surface area.

7. The absorbent article of claim 4 wherein said first section and said second section further comprise a respective transverse end wherein for disposal said absorbent article is folded substantially along said transverse axis so that said cover is folded upon itself and said open portion of said pocket device is folded over said transverse ends to retain the absorbent article in a folded condition.

8. The absorbent article of claim 1 wherein said open portion of said pocket device is expandable.

9. A sanitary napkin adapted to be adhesively secured to a wearer's body, said sanitary napkin comprising:
a. a fluid-permeable cover having a body facing surface;
b. a liquid-impermeable baffle having a garment-facing surface;
c. an absorbent positioned between said cover and said baffle wherein said cover and said baffle extend beyond an edge of said absorbent to form a perimeter of said sanitary napkin;
d. adhesive means for securing said absorbent article to the wearer's body, said adhesive means being affixed to said body facing surface; and
e. a pocket device having a fixed portion secured to said baffle at or inward from said perimeter and an open portion adapted for assisting removal of said absorbent article.

10. The sanitary napkin of claim 9 further having a transverse axis dividing the sanitary napkin into a first section and a second section of substantially equal surface area wherein said pocket device is secured to one of said sections and said pocket device covers greater than about 20 of said one section surface area.

11. The sanitary napkin of claim 10 wherein said pocket means covers greater than 50 percent of said one section surface area.

12. The sanitary napkin of claim 9 wherein said pocket device includes a means for expanding said open portion.

13. The sanitary napkin of claim 12 wherein said expanding means is a pleat, fold or elastomeric material.

14. The sanitary napkin of claim 12 wherein said expanding means increases a volume of the open portion by greater than about 5 percent.

15. The sanitary napkin of claim 14 wherein said expanding means increases said volume by greater than about 50 percent.

16. The sanitary napkin of claim 10 wherein each section has a respective transverse end and during disposal the sanitary napkin is folded substantially along said transverse axis and said open portion of said pocket means is folded over said transverse ends to retain the sanitary napkin in a folded condition.

17. A sanitary napkin adapted to be adhesively secured to a wearer's body, said sanitary napkin comprising:
a. a fluid-permeable cover having a body facing surface;
b. a liquid-impermeable baffle having a garment-facing surface;
c. an absorbent positioned between said cover and said baffle wherein said cover and said baffle extend beyond an edge of said absorbent to form a perimeter of said sanitary napkin;
d. adhesive means for securing said absorbent article to the wearer's body, said adhesive means being affixed to said body facing surface; and
e. a pocket device having a fixed portion secured to said baffle at or inward from said perimeter and an open portion adapted for assisting removal of said absorbent article, said sanitary napkin having a transverse axis dividing the sanitary napkin into a first section and a second section of substantially equal surface area, said pocket device being secured to one of said sections and covers greater than about 50 percent of said one section surface area.

18. The sanitary napkin of claim 17 wherein said pocket device includes a means for expanding said open portion thereby increasing a volume of said open portion by greater than about 5 percent.

19. The sanitary napkin of claim 18 wherein said expanding means is a pleat, fold or elastomeric material.

20. The sanitary napkin of claim 19 wherein said expanding means is expandable in at least one direction.

21. The sanitary napkin of claim 17 wherein each section has a respective transverse end and during disposal the sanitary napkin is folded substantially along said transverse axis and said open portion of said pocket means is folded over said transverse ends to retain the sanitary napkin in a folded condition.

22. A method of hygienically sealing a sanitary napkin for disposal, the sanitary napkin has a transverse axis and two transverse ends and is adapted to be secured to a wearer, said method comprising:
a) providing a sanitary napkin having a cover with a body facing surface, a baffle, an absorbent between the cover and the baffle, an adhesive secured to said body facing surface, and a pocket device, said pocket device having a fixed portion secured to said baffle and an open portion adapted for assisting removal of the sanitary napkin,
b) folding said sanitary napkin about said transverse axis whereby the cover is folded upon itself and the two transverse ends are substantially aligned with each other, and
c) rolling or folding the open portion of the pocket means over the transverse ends to retain the sanitary napkin in a folded condition.
